(19) United States
(12) Patent Application Publication
(10) Pub. No.: US 2026/0174198 A1
(43) Pub. Date: Jun. 25, 2026

(54) SHOCK-ABSORBING WEARABLE RING STRUCTURES

(71) Applicant: Oura Health Oy, Oulu (FI)

(72) Inventors: Heikki Juhani Huttunen, Haukipudas (FI); Marko Uusitalo, Oulu (FI); Jouni Juhani Huopana, Oulu (FI); Iida Paksuniemi, Oulu (FI)

(21) Appl. No.: 19/423,851

(22) Filed: Dec. 17, 2025

Related U.S. Application Data
(60) Provisional application No. 63/737,254, filed on Dec. 20, 2024.

(51) Int. Cl. A44C 9/00

(52) U.S. Cl. CPC A44C 9/0053; A44C 9/003

(57) ABSTRACT

A shock-absorbing wearable ring device is described. A wearable ring device includes an inner housing and one or more sensors configured to acquire physiological data from the user, where the sensors are at least partially encapsulated within a moldable material. The wearable ring device further includes an outer housing that surrounds the housing, where an inner surface of the outer housing and an outer surface of the moldable material are separated by a gap. The wearable ring device further includes side covers disposed between the outer housing and the inner housing on a lateral sides of the wearable ring device, where the side covers are configured to undergo a mechanical deformation in response to an external force exerted on the outer housing, and where a depth of the gap between the outer housing and moldable material changes based at least in part on the mechanical deformation of the side covers.

### CROSS REFERENCES

The present Application for Patent claims priority to U.S. Provisional Patent Application No. 63/737,254 by Huttunen et al., entitled “SHOCK ABSORBING WEARABLE RING STRUCTURES,” filed December 20, 2024, which is assigned to the assignee hereof and which is expressly incorporated by reference herein.

### FIELD OF TECHNOLOGY

The following relates to wearable devices and data processing, including shock-absorbing wearable ring structures.

### BACKGROUND

Some wearable devices may be configured to collect data from users to help the users understand more about their overall physiological health and well-being. However, wearable devices may be exposed to external forces while worn by the user, which may cause one or more components of the wearable devices to become loose or move in an unintentional manner, reducing a lifespan of the wearable device.

### BRIEF DESCRIPTION OF THE DRAWINGS

FIG. 1 shows an example of a manufacturing process for shock-absorbing wearable ring structures in accordance with aspects of the present disclosure.

FIG. 2 shows example wearable ring devices that exhibit shock-absorbing wearable ring structures in accordance with aspects of the present disclosure.

FIG. 3 shows an example of a wearable ring device that exhibits a shock-absorbing wearable ring structure in accordance with aspects of the present disclosure.

FIGS. 4 and 5 illustrate examples of systems that support shock-absorbing wearable ring structures in accordance with aspects of the present disclosure.

FIG. 6 shows a flowchart illustrating methods that support shock-absorbing wearable ring structures in accordance with aspects of the present disclosure.

### DETAILED DESCRIPTION

Wearable devices (e.g., rings, watches, etc.) may be used to measure biometric data of a user and report the data to the user. Wearable devices may be manufactured with various materials for both functional and aesthetic purposes. For example, some wearable ring devices are manufactured with metallic outer shells that couple with inner shells of the ring to seal the electronic components of the wearable ring device between the inner and outer shells. The electronic components may be encapsulated within an epoxy material (or other moldable material) that is sealed by the inner/outer shells. The metallic outer shells may be able to flex when the ring is exposed to an external force (e.g., when the ring is dropped), which dissipates the force and prevents the force from being transferred to the epoxy and electronic components, thereby making the ring more durable and preventing the electronic components from being damaged. However, such metallic materials may be susceptible to scratches, which may diminish the aesthetic appearance of the ring. Comparatively, other materials that are scratch resistant, such as ceramic materials, may be unable to flex when the ring is dropped or exposed to other external forces. Without some degree of flex, these ceramic outer shells may not dissipate the external force and may instead transfer the force to the inner epoxy of the ring, which increases the likelihood that the electronic components will be damaged.

Accordingly, aspects of the present disclosure are directed to shock-absorbing wearable ring devices that are able to protect the internal components and sensors from damage from external forces. In particular, aspects of the present disclosure are directed to manufacturing techniques for wearable ring devices that utilize air gaps between the outer shell and the internal components of the ring to help prevent damage to the electronic components. Such techniques may enable wearable ring devices to be manufactured with outer shells made from inflexible materials (e.g., ceramic outer shells), while simultaneously preventing such inflexible outer shells from transferring external forces to the inner epoxy (and/or other inner components, such as the sensors/electronic components) that may damage the ring.

For example, electronic components (e.g., sensors) of the wearable device may be attached to an inner ring-shaped housing (e.g., inner shell) of the wearable ring device via an epoxy molding process, thereby forming a “ring assembly.” Subsequently, an outer ring-shaped housing (e.g., outer shell) of the wearable ring device may be coupled to the ring assembly (e.g., the inner ring-shaped housing/epoxy) such that an air gap exists between the molded epoxy (which encapsulates the electronic components) and the outer ring-shaped housing. The air gap may prevent any external forces applied to the outer ring-shaped housing (such as forces resulting from the user dropping the ring) from being transferred to the molded epoxy/electronic components. For instance, the outer ring-shaped housing may be attached using “side covers” on the lateral sides of the ring, where external forces exerted on the outer ring-shaped housing are transferred through the side covers, rather than to the epoxy and electronic components. In such cases, the side covers may include ring-shaped fittings, a cured adhesive material, or both. In some cases, the air gap may extend 360° around the perimeter (e.g., circumference) of the ring. In additional or alternative cases, the air gap may be filled with a foam or other compressible material for additional shock absorbance.

Aspects of the present disclosure are initially described in the context of an example manufacturing process and example wearable ring devices. Additional aspects of the disclosure are initially described in the context of systems supporting physiological data collection from users via wearable devices. Aspects of the disclosure are further illustrated by and described with reference to apparatus diagrams, system diagrams, and flowcharts that relate to shock-absorbing wearable ring structures.

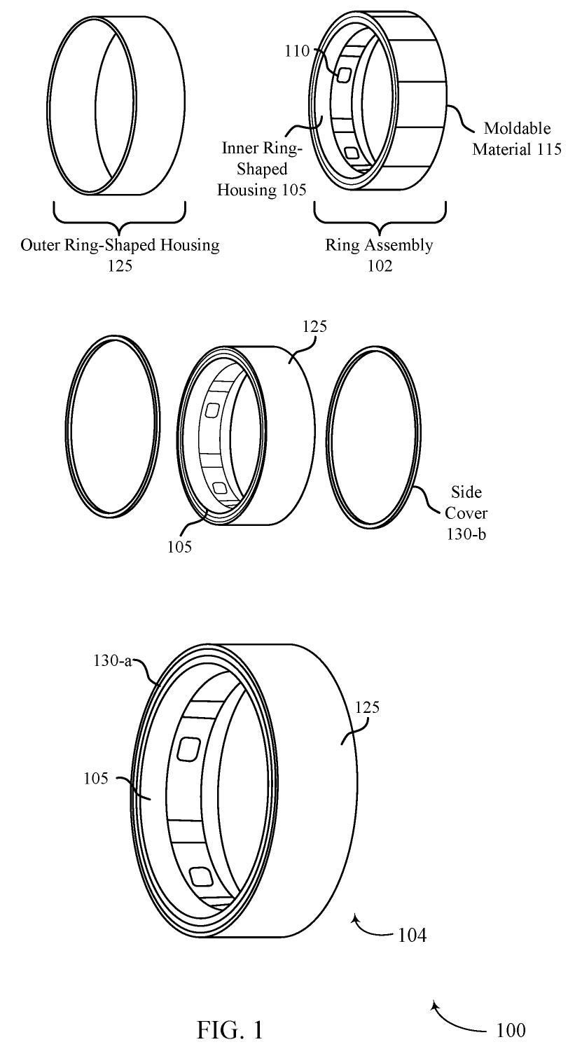

FIG. 1 shows an example of a manufacturing process 100 for shock-absorbing wearable ring structures in accordance with aspects of the present disclosure. In particular, the manufacturing process 100 may include an example for manufacturing shock-absorbing wearable ring devices 104, as described herein.

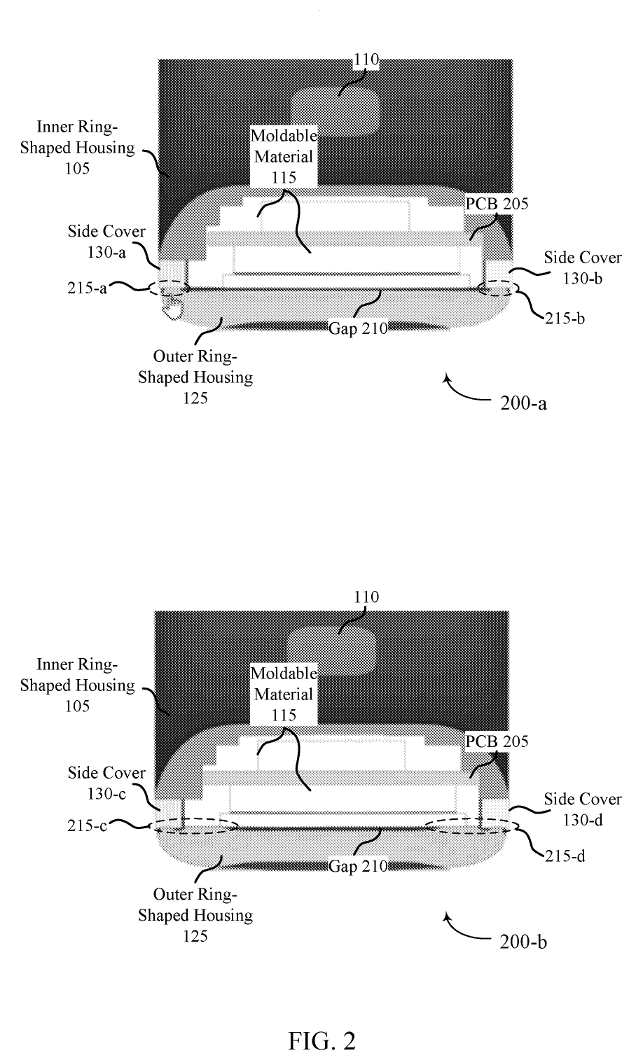

In some aspects, the electrical components of the wearable ring device 104 (e.g., PCB, sensors, battery) may be attached to an inner ring-shaped housing 105 (e.g., inner shell, inner cover) of the wearable ring device 104. Subsequently, the inner ring-shaped housing 105 (e.g., and attached electrical components) may be placed into a mold, such that a moldable material 115, such as a clear epoxy, may be injected into the mold to secure the electrical components to the inner ring-shaped housing 105. In this regard, as shown in FIG. 2, the PCB, sensors, and/or battery may be at least partially (e.g., fully) encapsulated or covered by the moldable material 115. Additionally, the molding procedure (e.g., injection molding procedure) may cause the moldable material 115 to fill one or more apertures 110 of the inner ring-shaped housing 105, such that the one or more optical sensors may be secured with relation to the apertures 110 to enable data collection. For example, the moldable material 115 may be configured to form domes or protrusions that fill/cover the apertures and extend from the inner curved surface of the inner ring-shaped housing 105, where the sensors are configured to acquire data through the apertures and domes/protrusions. That is, the sensors and other optical components of the wearable ring device 104 may be configured to acquire physiological data from the user through the one or more apertures 110.

In this regard, the molding procedure may be used to secure the electrical components (e.g., PCB, sensors, battery) of the wearable ring device 104 to the inner ring-shaped housing 105. The structure resulting from the molding procedure may be referred to as a ring assembly 102 (e.g., ring engine assembly), which includes the inner ring-shaped housing 105, the PCB/sensors, and the cured moldable material 115 (where the PCB, sensors, and battery are encapsulated within the moldable material 115). In this regard, the inner ring-shaped housing 105 may include or define an inner curved surface (e.g., inner circumferential surface) of the ring assembly 102 and wearable ring device 104, where the moldable material 115 includes or defines an outer surface of the ring assembly 102. The ring assembly 102 may essentially be an operational wearable ring device 104 without an outer ring-shaped housing 125 (e.g., outer shell, outer cover).

Subsequently, the outer ring-shaped housing 125 be placed around the ring assembly 102 such that the outer ring-shaped housing 125 at least partially surrounds the inner ring-shaped housing 105/ring assembly 102. For example, the outer ring-shaped housing 125 may extend around a full perimeter (e.g., full circumference) of the wearable ring device 104, as shown in FIG. 1. In this regard, the outer ring-shaped housing 125 may include or define an outer curved surface (e.g., outer circumferential surface) of the wearable ring device 104.

In some aspects, the outer ring-shaped housing 125 may be secured to the ring assembly 102 (e.g., secured to the inner ring-shaped housing 105) using one or more side covers 130 (e.g., side cover 130-a, side cover 130-b) on the lateral sides of the wearable ring device 104. In some cases, the side covers 130 may include ring-shaped fittings that are inserted into the slots formed between the inner ring-shaped housing 105 and the outer ring-shaped housing 125. In such cases, the ring-shaped fittings (e.g., side covers 130) may be slightly wider than the slots between the outer ring-shaped housing 125 and the inner ring-shaped housing 105, such that the ring-shaped fittings mechanically deform upon being inserted, or pressed, into the slots due to an applied force. In such cases, the mechanical deformation of the ring-shaped fittings may cause the ring-shaped fittings (e.g., side covers) to engage with one or more mechanical locking features on the outer ring-shaped housing 125, the inner ring-shaped housing 105, or both. Additionally, or alternatively, each of the ring-shaped fittings (e.g., side covers 130) may include one or more flanges, or locking wings, to enable the ring-shaped fittings to engage the one or more mechanical locking features on the outer ring-shaped housing 125, the inner ring-shaped housing 105, or both.

In other cases, the side covers 130 may be formed by pouring an adhesive material (e.g., UV glue) into the slots formed between the inner ring-shaped housing 105 and the outer ring-shaped housing 125. That is, in some cases, heat or light sensitive glue may be used to form the side covers 130. For example, UV glue may be applied to the slots (e.g., and any gaps) between the outer ring-shaped housing 125 and the inner ring-shaped housing 105, such that the UV glue fills the entirety of the slots. In such cases, the UV light may be applied directly to the UV glue to cure the UV glue, such that the UV glue solidifies within the slots, thereby forming the side covers 130 and locking the outer ring-shaped housing 125 to the inner ring-shaped housing 105. In additional or alternative implementations, a self-curing glue or composite may also be used. Such self-curing materials may be cured by mixing components that enable polymerization, or through exposure to air. In other cases, the side covers 130 may be formed using a heat-cured material.

As noted previously herein, some wearable devices may utilize metallic materials for the outer ring-shaped housing 125. Such metallic outer shells may be able to flex when the wearable ring device 104 is exposed to an external force (e.g., when the wearable ring device 104 is dropped), which dissipates the force and prevents the force from being transferred to the moldable material 115 and the electronic components (e.g., PCB, battery, sensors) encapsulated within the moldable material 115, thereby making the wearable ring device 104 more durable and preventing the electronic components from being damaged. However, such metallic materials may be susceptible to scratches, which may diminish the aesthetic appearance of the wearable ring device 104. Comparatively, other materials that are scratch resistant, such as ceramic materials, may be unable to flex when the ring is dropped or exposed to other external forces. Without some degree of flex, these ceramic outer shells (e.g., outer ring-shaped housing 125 made of ceramic) may not dissipate the external force and may instead transfer the force to the moldable material 115 (and electronic components encapsulated within the moldable material 115), which increases the likelihood that the electronic components will be damaged.

Accordingly, aspects of the present disclosure are directed to shock-absorbing wearable ring devices 104 that are able to protect the internal components and sensors from damage from external forces. In particular, the manufacturing process 100 shown and described in FIG. 1 may be used to manufacture the wearable ring device 104 with a gap between the outer ring-shaped housing 125 and the ring assembly 102/moldable material 115 to help prevent damage to the electronic components. Such techniques may enable the wearable ring device 104 to be manufactured with an outer ring-shaped housing 125 made from inflexible materials (e.g., ceramic), while simultaneously preventing such inflexible outer shells from transferring external forces to the moldable material 115 (and electronic components encapsulated within the moldable material 115), thereby preventing damage to the wearable ring device 104.

In some cases, the gap between the outer ring-shaped housing 125 and the moldable material 115/ring assembly 102 may extend 360° around the perimeter/circumference of the wearable ring device 104. In additional or alternative cases, the gap may be filled with a foam or other compressible material for additional shock absorbance. By implementing a gap between the outer ring-shaped housing 125 and the moldable material 115/ring assembly 102, external forces exerted on the outer ring-shaped housing 125 (e.g., external forces caused by dropping the ring) may be transferred through the side covers 130, rather than to the moldable material 115 and electronic components. In some aspects, the side covers 130 may be able to deform in response to external forces exerted on the outer ring-shaped housing 125, thereby dissipating the force. In such cases, the gap between the ring-shaped housing 125 and the moldable material 115 may change (e.g., compress, or otherwise reduce the size of the gap) based on the deformation of the side covers 130, thereby preventing the external force from being transferred to the moldable material 115 and the electronic components. For example, when an external force is exerted on the outer ring-shaped housing 125 (such as a force due to dropping the ring), the outer ring-shaped housing 125 may transfer the external force to the side covers 130, causing the side covers to compress (e.g., undergo a mechanical deformation). The compression of the side covers 130 may result in the outer ring-shaped housing 125 moving toward the inner ring-shaped housing 105, thereby reducing the depth of the gap between the outer ring-shaped housing 125 and the moldable material 115/electronic components.

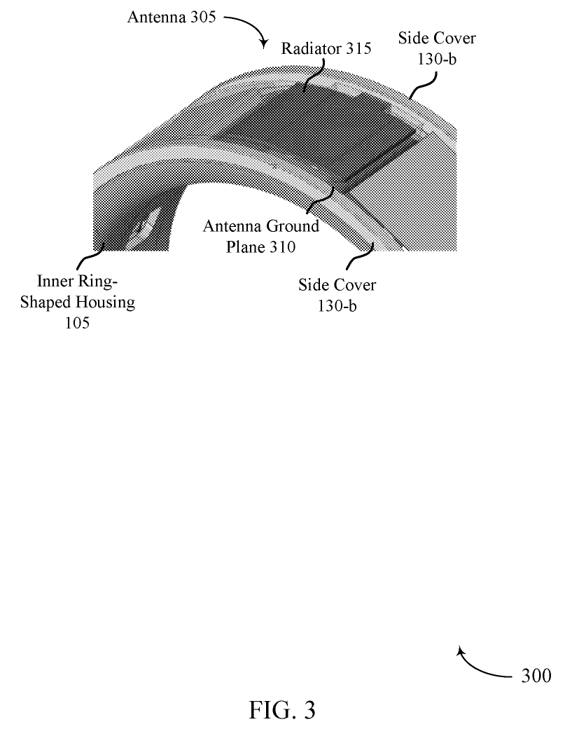

Attendant advantages of the manufacturing techniques and shock-absorbing ring form factors described herein are further shown and described with reference to FIGS. 2 and 3.

FIG. 2 shows examples of wearable ring devices 200-a, 200-b that exhibit shock-absorbing wearable ring structures in accordance with aspects of the present disclosure. Aspects of the wearable ring devices 200-a, 200-b may implement, or be implemented by, the manufacturing process 100 in FIG. 1. In particular, FIG. 2 may illustrate example cross-sectional views of the wearable ring device 104 shown and described in FIG. 1.

As described previously herein, the wearable ring devices 200-a, 200-b may include an inner ring-shaped housing 105 and an outer ring-shaped housing 125. Electronic components (e.g., PCB 205, battery, sensors) of the wearable ring devices 200 may be disposed between the inner ring-shaped housing 105 and the outer ring-shaped housing 125. For example, as shown and described in FIG. 1, the electronic components (e.g., PCB 205, battery, sensors) may be coupled to the inner ring-shaped housing 105 using a moldable material 115. In such cases, the electronic components may be at least partially (e.g., completely) encapsulated within the moldable material 115, as shown in FIG. 2.

Further, as shown and described in FIG. 2, the structure resulting from the molding procedure may be referred to as a ring assembly 102 (e.g., ring engine assembly), which includes the inner ring-shaped housing 105, the PCB 205/sensors, and the cured moldable material 115 (where the PCB 205, sensors, and battery are encapsulated within the moldable material 115). In this regard, the inner ring-shaped housing 105 may include or define an inner curved surface (e.g., inner circumferential surface) of the ring assembly 102 and wearable ring device 104, where the moldable material 115 includes or defines an outer surface of the ring assembly 102.

The outer ring-shaped housing 125 may at least partially surround the inner ring-shaped housing 105/ring assembly 102. For example, the outer ring-shaped housing 125 may extend around a full circumference of the wearable ring device 200. The outer ring-shaped housing 125 may be made from one or more materials, such as metallic materials, ceramic materials, and the like.

In some aspects, the outer ring-shaped housing 125 may be secured to the ring assembly 102 (e.g., secured to the inner ring-shaped housing 105) using one or more side covers 130 (e.g., side cover 130-a, side cover 130-b) on the lateral sides of the wearable ring device 104. In some cases, the side covers 130 may include ring-shaped fittings that are inserted into the slots formed between the inner ring-shaped housing 105 and the outer ring-shaped housing 125. In other cases, the side covers 130 may be formed by pouring an adhesive material (e.g., UV glue) into the slots formed between the inner ring-shaped housing 105 and the outer ring-shaped housing 125. That is, in some cases, heat or light sensitive glue may be used to form the side covers 130.

In some aspects, the outer ring-shaped housing 125 may be separated from the ring assembly 102/moldable material by a gap 210. For example, as shown in FIG. 2, an inner surface of the outer ring-shaped housing 125 and an outer surface of the moldable material 115 may be separated by a gap 210. In some aspects, the gap 210 may extend at least a portion around a circumference of the wearable ring device 200. For example, in some cases, the gap 210 may extend 360° around the circumference of the wearable ring device 200.

In some aspects, the gap 210 between the outer ring-shaped housing 125 and the moldable material 115/ring assembly 102 may span at least a portion of a width of the wearable ring device 200 between a first lateral side and a second lateral side of the wearable ring device 200. For example, as shown in the first wearable ring device 200-a illustrated in the top diagram of FIG. 2, the outer ring-shaped housing 125 may be coupled to the wearable ring device 200-a via a first contact point 215-a with the first side cover 130-a and a second contact point 215-b with the second side cover 130-b, where the gap 210 spans the portion of the width of the wearable ring device 200-a between the first contact point 215-a and the second contact point 215-b. In this regard, in the context of the first wearable ring device 200-a illustrated in the top diagram of FIG. 2, the gap 210 may completely separate the moldable material 115 and the outer ring-shaped housing 125 such that there is no direct contact between the outer ring-shaped housing 125 and the moldable material 115. In this example, the inner surface of the outer ring-shaped housing 125 (e.g., inner surface that faces the gap 210 and moldable material 115) may be substantially flat, where the outer ring-shaped housing 125 includes flanges or protrusions which contact the side covers 130-a, 130-b on the lateral sides.

By way of another example, as shown in the second wearable ring device 200-b illustrated in the bottom diagram of FIG. 2, the outer ring-shaped housing 125 may be coupled to the wearable ring device 200-a via a first contact point 215-c with the first side cover 130-a and a first portion of the moldable material 115, and a second contact point 215-d with the second side cover 130-b and a second portion of the moldable material 115. That is, as compared to the first wearable ring device 200-a in which the outer ring-shaped housing 125 is completely separated from the moldable material 115, the outer ring-shaped housing 125 of the second wearable ring device 200-b may partially contact the outer portions of the moldable material 115.

As noted previously herein, the gap 210 may be used to provide shock-absorbance for the wearable ring device 200. That is, the gap 210 may prevent external forces exerted on the outer ring-shaped housing 125 from being transferred to the moldable material 115 and electronic components (e.g., PCB 205, battery, sensors). Stated differently, by implementing a gap between the outer ring-shaped housing 125 and the moldable material 115/ring assembly 102, external forces exerted on the outer ring-shaped housing 125 (e.g., external forces caused by dropping the ring) may be transferred through the side covers 130 (e.g., glue, adhesive, ring-shaped fittings), rather than directly to the moldable material 115 and electronic components. In some implementations, the gap 210 may be filled with a foam or other compressible material for additional shock absorbance.

In some aspects, the side covers 130 may be able to deform in response to external forces exerted on the outer ring-shaped housing 125, thereby dissipating the external force applied to the outer ring-shaped housing 125. In other words, kinetic energy may be absorbed by the side covers 130 through the deformation of the side covers. In particular, due to the existence of the gap 210, an external force exerted on the outer ring-shaped housing 125 may be transferred to the side covers 130 and/or the inner ring-shaped housing 105, rather than directly to the moldable material 115. Stated differently, the gap 210 and/or the side covers 130 may enable the wearable ring devices 200 to absorb the impact of shocks/external forces, thereby making the electronic components of the wearable rind devices 200 more resistant (e.g., less vulnerable) to the external forces.

The ability of the side covers 130 to deform may enable the wearable ring device 104 to dissipate external forces and protect the internal electronic components even in cases where the outer ring-shaped housing 125 is formed from an inflexible material, such as ceramic. The side covers 130 may be manufactured from a material that is elastically deformable, such that the side covers 130 are able to be deformed (e.g., compressed) in response to an external force exerted on the outer ring-shaped housing 125, and subsequently return to their original shape and size (e.g., expand back to the original shape/size).

In some aspects, a width of the gap 210 between the ring-shaped housing 125 and the moldable material 115 may change (e.g., compress) based on the deformation of the side covers 130, thereby preventing the external force from being transferred to the moldable material 115 and the electronic components. For example, when an external force is exerted on the outer ring-shaped housing 125 (such as a force due to dropping the ring), the outer ring-shaped housing 125 may transfer the external force to the side covers 130, causing the side covers 130 to compress (e.g., undergo a mechanical deformation). The compression of the side covers 130 may result in the outer ring-shaped housing 125 moving toward the inner ring-shaped housing 105, thereby reducing the depth of the gap 210 between the outer ring-shaped housing 125 and the moldable material 115/electronic components.

In some aspects, the magnitude of the deformation of the side covers 130 may be configured (e.g., by selection of the size and/or material of the side covers 130) such that the corresponding deformation of the gap 210 does not enable the inner surface of the outer ring-shaped housing 125 to contact the outer surface of the moldable material 115. The dimensions of the gap 210 (both without any exerted external force and after application of external forces) may be determined by simulations.

Referring to the first wearable ring device 200-a, the gap 210 may completely separate the outer ring-shaped housing 125 and the moldable material 115. As such, an entirety of an external force exerted on the outer ring-shaped housing 125 may be transferred/dissipated through the side covers 130 via the contact points 215-a, 215-b.

Comparatively, in the context of the second wearable ring device 200-b, the outer ring-shaped housing 125 may partially contact portions of the moldable material 115 towards the lateral sides of the ring at the contact points 215-c, 215-d. However, the existence of the gap 210 in the second wearable ring device 200-b may still protect the electronic components of the wearable ring device 200-b compared to some conventional devices. In particular, the gap 210 of the second wearable ring device 200-b may prevent direct contact between the outer ring-shaped housing 125 and the inner-most portion of the moldable material 115, which may include the most fragile and sensitive components/sensors of the device. That is, the portions of the moldable material 115 toward the lateral sides and contact points 215-c, 215-d may be more durable as compared to the inner-most portions of the moldable material 115. Further, the second wearable ring device 200-b may be manufactured such that there are not any sensitive electrical components disposed within the portions of the moldable material 115 proximate to the contact points 215-c, 215-d. Thus, as compared to some conventional devices, the existence of the gap 210 within both the first wearable ring device 200-a and the second wearable ring device 200-b may protect the electrical components of the respective devices from damage.

As noted previously herein, the use of a ceramic material for the outer ring-shaped housing 125 may make the outer ring-shaped housing 125 more resistant to scratches as compared to other materials such as metal. Additionally, using a non-conductive material such as ceramic for the outer ring-shaped housing 125 may prevent the wearable ring devices 200 from exhibiting a “capacitive effect” that may result from metallic materials. For example, in cases where both the inner ring-shaped housing 105 and the outer ring-shaped housing 125 are formed from conductive materials (e.g., metallic materials), the respective housings may develop electrical charges, thereby resulting in an electrostatic potential difference between the inner ring-shaped housing 105 and the outer ring-shaped housing 125. Such differences in electrostatic potential may lead to electrostatic discharge, which may damage the internal components (e.g., PCB 205). Comparatively, using a non-conductive material such as ceramic for the outer ring-shaped housing 125 may prevent the outer ring-shaped housing 125 from carrying/developing an electrical charge, thereby preventing or otherwise minimizing electrostatic discharge.

Additionally, the use of a non-conductive material such as ceramic for the outer ring-shaped housing 125 may improve wireless communications capabilities of the wearable ring devices 200, as is further shown and described with reference to FIG. 3.

FIG. 3 shows an example of a wearable ring device 300 that exhibits a shock-absorbing wearable ring structure in accordance with aspects of the present disclosure. Aspects of the wearable ring device 300 may implement, or be implemented by, the manufacturing process 100 in FIG. 1, the wearable ring devices 200-a, 200-b in FIG. 2, or both. In particular, FIG. 3 may illustrate example perspective internal view of the wearable ring devices 104, 200-a, 200-b shown and described in FIGS. 1-2.

As described previously herein, the wearable ring device 300 may include an inner ring-shaped housing 105 and an outer ring-shaped housing 125 (not shown in FIG. 3), where the inner ring-shaped housing 105 and the outer ring-shaped housing 125 are coupled together using one or more side covers 130-a, 130-b. The side covers 130-a, 130-b may include ring-shaped fittings, molded/cured adhesive material (e.g., UV glue), or both).

As shown in FIG. 3, the wearable ring device 300 may include an antenna 305. In some aspects, the antenna 305 may be encapsulated within the moldable material 115, as described herein. The wearable ring device 300 may utilize the antenna 305 to perform wireless communications with other devices, such as a user device (e.g., smart phone). For example, the wearable device 300 may use the antenna 305 to exchange information, such as physiological data collected by the wearable device 300, with the user device.

In some wearable devices, the antenna may be a trace antenna that is formed by conductive traces on a circuit board that is disposed along a circumferential portion of the wearable device. Together with an antenna ground plane (which may be the ground plane of the circuit board and/or a metal chassis of the wearable device), the trace antenna may generate an electromagnetic field that the wearable device uses for wireless communications.

The antenna 305 may include an antenna ground plane 310 and a radiator 315. The antenna ground plane 310 may include a conductive material (e.g., metallic material). For example, antenna ground plane 310 may include the ground plane of a circuit board (e.g., PCB 205) within the wearable ring device 300, may include an inner metal surface (e.g., a metal chassis) within the wearable device 300 (or, functionally speaking, may be considered to be both). In some aspects, the antenna ground plane 310 may be positioned adjacent to (e.g., in contact with) the inner ring-shaped housing 105. For example, the antenna ground plane 310 may be curved and extend along the curvature of the interior sidewall of the inner ring-shaped housing 105. The antenna ground plane 325 may be configured to reflect an electromagnetic field that is generated by the antenna 305/radiator 315 when the antenna 305 is energized.

In some aspects, the radiator 315 of the antenna 305 may be configured to generate an electromagnetic field that is used to facilitate wireless communications. The radiator 315 may be positioned such that it overlaps with the antenna ground plane 310, thereby enabling an electromagnetic field to be generated between the radiator 315 and the antenna ground plane 310.

In some conventional wearable ring devices with metallic outer shells/covers (e.g., metallic outer ring-shaped housing 125), the metallic outer shells may prevent, inhibit, or otherwise interfere with wireless communications. In such cases with metallic outer shells, the antenna 305 may be configured to transmit/receive wireless signals through the side covers 130 (as opposed to transmitting/receiving wireless signals through the metallic outer cover). Comparatively, by utilizing a non-conductive outer ring-shaped housing 125, the antenna 305 may be able to transmit and receive wireless signals through the ceramic outer ring-shaped housing 125. As such, techniques described herein that enable the outer cover to be manufactured with non-metallic materials may also increase the strength and reliability of wireless communications performed by the wearable ring device 300.

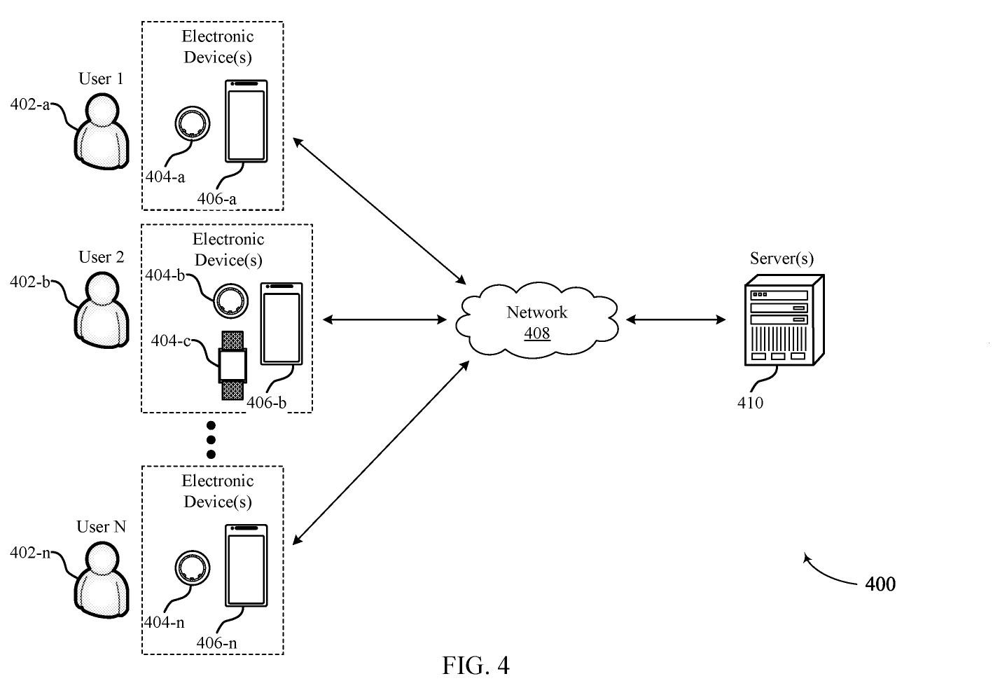

FIG. 4 illustrates an example of a system 400 that supports shock-absorbing wearable ring structures in accordance with aspects of the present disclosure. The system 400 includes a plurality of electronic devices (e.g., wearable devices 404, user devices 406) that may be worn and/or operated by one or more users 402. The system 400 further includes a network 408 and one or more servers 410.

The electronic devices may include any electronic devices known in the art, including wearable devices 404 (e.g., ring wearable devices, watch wearable devices, etc.), user devices 406 (e.g., smartphones, laptops, tablets). The electronic devices associated with the respective users 402 may include one or more of the following functionalities: 1) measuring physiological data, 2) storing the measured data, 3) processing the data, 4) providing outputs (e.g., via GUIs) to a user 402 based on the processed data, and 5) communicating data with one another and/or other computing devices. Different electronic devices may perform one or more of the functionalities.

Example wearable devices 404 may include wearable computing devices, such as a ring computing device (hereinafter “ring”) configured to be worn on a user’s 402 finger, a wrist computing device (e.g., a smart watch, fitness band, or bracelet) configured to be worn on a user’s 402 wrist, and/or a head mounted computing device (e.g., glasses/goggles). Wearable devices 404 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 404 may also be attached to, or included in, articles of clothing. For example, wearable devices 404 may be included in pockets and/or pouches on clothing. As another example, wearable device 404 may be clipped and/or pinned to clothing, or may otherwise be maintained within the vicinity of the user 402. Example articles of clothing may include, but are not limited to, hats, shirts, gloves, pants, socks, outerwear (e.g., jackets), and undergarments. In some implementations, wearable devices 404 may be included with other types of devices such as training/sporting devices that are used during physical activity. For example, wearable devices 404 may be attached to, or included in, a bicycle, skis, a tennis racket, a golf club, and/or training weights.

Much of the present disclosure may be described in the context of a wearable device 404, which may include finger-worn wearable devices, wrist-worn wearable devices, and the like. Accordingly, the terms “wearable device 404,” “wearable ring device,” “ring,” and like terms, may be used interchangeably, unless noted otherwise herein. However, the use of the terms “wearable ring device” and/or “ring” are not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 406 may include handheld mobile computing devices, such as smartphones and tablet computing devices. User devices 406 may also include personal computers, such as laptop and desktop computing devices. Other example user devices 406 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 406 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 404, user devices 406) may measure physiological parameters of respective users 402, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 404), mobile device application, or a server computing device may process received physiological data that was measured by other devices.

In some implementations, a user 402 may operate, or may be associated with, multiple electronic devices, some of which may measure physiological parameters and some of which may process the measured physiological parameters. In some implementations, a user 402 may have a ring (e.g., wearable device 404) that measures physiological parameters. The user 402 may also have, or be associated with, a user device 406 (e.g., mobile device, smartphone), where the wearable device 404 and the user device 406 are communicatively coupled to one another. In some cases, the user device 406 may receive data from the wearable device 404 and perform some/all of the calculations described herein. In some implementations, the user device 406 may also measure physiological parameters described herein, such as motion/activity parameters.

For example, as illustrated in FIG. 4, a first user 402-a (User 1) may operate, or may be associated with, a wearable device 404-a (e.g., wearable ring device) and a user device 406-a that may operate as described herein. In this example, the user device 406-a associated with user 402-a may process/store physiological parameters measured by the wearable device 404-a. Comparatively, a second user 402-b (User 2) may be associated with wearable devices 404-b and 404-c (e.g., wearable ring device and a wrist-worn wearable device, such as a watch) and a user device 406-b, where the user device 406-b associated with user 402-b may process/store physiological parameters measured by the wearable devices 404-b and 404-c. Moreover, an nth user 402-n (User N) may be associated with an arrangement of electronic devices described herein (e.g., wearable device 404-n, user device 406-n). In some aspects, wearable devices 404 (e.g., wearable ring devices, wrist-worn wearable devices) and other electronic devices may be communicatively coupled to the user devices 406 of the respective users 402 via Bluetooth, Wi-Fi, and other wireless protocols. Moreover, in some cases, the wearable device 404 and the user device 406 may be included within (or make up) the same device. For example, in some cases, the wearable device 404 may be configured to execute an application associated with the wearable device 404, and may be configured to display data via a GUI.

In some implementations, the wearable devices 404 (e.g., wearable ring devices) of the system 400 may be configured to collect physiological data from the respective users 402 based on arterial blood flow within the user’s finger. In particular, a wearable ring device may utilize one or more light-emitting components, such as LEDs (e.g., red LEDs, green LEDs) that emit light on the palm-side of a user’s finger to collect physiological data based on arterial blood flow within the user’s finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 400 may be configured to collect physiological data from the respective users 402 based on blood flow diffused into a microvascular bed of skin with capillaries and arterioles. For example, the system 400 may collect PPG data based on a measured amount of blood diffused into the microvascular system of capillaries and arterioles. In some implementations, the wearable device 404 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the wearable device 404 (e.g., around an inner surface of the wearable ring device) has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a wearable ring device has been found to exhibit superior performance as compared to wearable devices worn on the wrist, as the wearable ring device may have greater access to arteries (as compared to capillaries), thereby resulting in stronger signals and more valuable physiological data.

The electronic devices of the system 400 (e.g., user devices 406, wearable devices 404) may be communicatively coupled to one or more servers 410 via wired or wireless communication protocols. For example, as shown in FIG. 4, the electronic devices (e.g., user devices 406) may be communicatively coupled to one or more servers 410 via a network 408. The network 408 may implement transfer control protocol and internet protocol (TCP/IP), such as the Internet, or may implement other network 408 protocols. Network connections between the network 408 and the respective electronic devices may facilitate transport of data via email, web, text messages, mail, or any other appropriate form of interaction within a computer network 408. For example, in some implementations, the wearable device 404-a associated with the first user 402-a may be communicatively coupled to the user device 406-a, where the user device 406-a is communicatively coupled to the servers 410 via the network 408. In additional or alternative cases, wearable devices 404 (e.g., wearable ring devices, wrist-worn wearable devices such as watches) may be directly communicatively coupled to the network 408.

The system 400 may offer an on-demand database service between the user devices 406 and the one or more servers 410. In some cases, the servers 410 may receive data from the user devices 406 via the network 408, and may store and analyze the data. Similarly, the servers 410 may provide data to the user devices 406 via the network 408. In some cases, the servers 410 may be located at one or more data centers. The servers 410 may be used for data storage, management, and processing. In some implementations, the servers 410 may provide a web-based interface to the user device 106 via web browsers.

In some aspects, the system 400 may detect periods of time that a user 402 is asleep, and classify periods of time that the user 402 is asleep into one or more sleep stages (e.g., sleep stage classification). For example, as shown in FIG. 4, User 402-a may be associated with a wearable device 404-a (e.g., wearable ring device) and a user device 406-a. In this example, the wearable device 404-a may collect physiological data associated with the user 402-a, including temperature, heart rate, HRV, respiratory rate, and the like. In some aspects, data collected by the wearable device 404-a may be input to a machine learning classifier, where the machine learning classifier is configured to determine periods of time that the user 402-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 402-a via a GUI of the user device 406-a. Sleep stage classification may be used to provide feedback to a user 402-a regarding the user’s sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 400 may utilize circadian rhythm-derived features to further improve physiological data collection, data processing procedures, and other techniques described herein. The term circadian rhythm may refer to a natural, internal process that regulates an individual’s sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 402-a via the wearable device 404-a. In this example, the circadian rhythm adjustment model may be configured to “weight,” or adjust, physiological data collected throughout a user’s natural, approximately 24-hour circadian rhythm. In some implementations, the system may initially start with a “baseline” circadian rhythm adjustment model, and may modify the baseline model using physiological data collected from each user 402 to generate tailored, individualized circadian rhythm adjustment models that are specific to each respective user 402.

In some aspects, the system 400 may utilize other biological rhythms to further improve physiological data collection, analysis, and processing by phase of these other rhythms. For example, if a weekly rhythm is detected within an individual’s baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

It should be appreciated by a person skilled in the art that one or more aspects of the disclosure may be implemented in a system 400 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

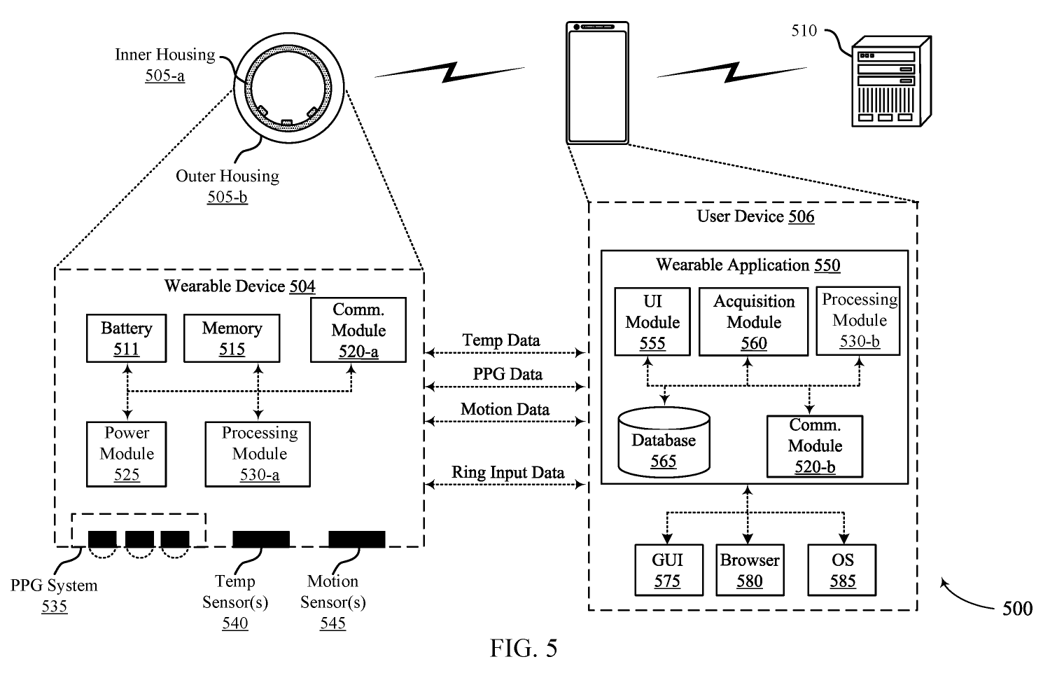

FIG. 5 illustrates an example of a system 500 that supports shock-absorbing wearable ring structures in accordance with aspects of the present disclosure. The system 500 may implement, or be implemented by, system 400. In particular, system 500 illustrates a wearable device 504 (e.g., wearable ring device), a user device 506, and a server 510, as described with reference to FIG. 4.

In some aspects, the wearable device 504 (e.g., wearable ring device) may be configured to be worn around a user’s finger, and may determine one or more user physiological parameters when worn around the user’s finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 500 further includes a user device 506 (e.g., a smartphone) in communication with the wearable device 504. For example, the wearable device 504 may be in wireless and/or wired communication with the user device 506. In some implementations, the wearable device 504 may send measured and processed data (e.g., temperature data, photoplethysmogram (PPG) data, motion/accelerometer data, ring input data, and the like) to the user device 506. The user device 506 may also send data to the wearable device 504, such as firmware/configuration updates. The user device 506 may process data. In some implementations, the user device 506 may transmit data to the server 510 for processing and/or storage.

The wearable device 504 may include a housing 505 that may include an inner housing 505-a and an outer housing 505-b. In some aspects, the inner housing 505-a, the outer housing 505-b, or both, may include a curved profile/surface. In particular, the housing 505 may exhibit any curved or “circumferential” profile, including a circular profile, an elliptical profile, and the like. Moreover, in some cases, the inner housing 505-a, the outer housing 505-b, or both, may include both curved (e.g., “circumferential”) and flat/planar portions. For the purposes of the present disclosure, the term “circumferential” may be used interchangeably with the term “curved” to refer to circular-shaped, elliptical-shaped, or other curved-shaped profile.

In some aspects, the housing 505 of the wearable device 504 may store or otherwise include various components of the ring including, but not limited to, device electronics, a power source (e.g., battery 511, and/or capacitor), one or more substrates (e.g., printable circuit boards) that interconnect the device electronics and/or power source, and the like. The device electronics may include device modules (e.g., hardware/software), such as: a processing module 530-a, a memory 515, a communication module 520-a, a power module 525, and the like. The device electronics may also include one or more sensors. Example sensors may include one or more temperature sensors 540, a PPG sensor assembly (e.g., PPG system 535), and one or more motion sensors 545.

The sensors may include associated modules (not illustrated) configured to communicate with the respective components/modules of the wearable device 504, and generate signals associated with the respective sensors. In some aspects, each of the components/modules of the wearable device 504 may be communicatively coupled to one another via wired or wireless connections. Moreover, the wearable device 504 may include additional and/or alternative sensors or other components that are configured to collect physiological data from the user, including light sensors (e.g., LEDs), oximeters, and the like.

The wearable device 504 shown and described with reference to FIG. 5 is provided solely for illustrative purposes. As such, the wearable device 504 may include additional or alternative components as those illustrated in FIG. 5. Additional or alternative wearable devices 504 that provide functionality described herein may be fabricated. For example, wearable devices 504 with fewer components (e.g., sensors) may be fabricated. In a specific example, a wearable device 504 with a single temperature sensor 540 (or other sensor), a power source, and device electronics configured to read the single temperature sensor 540 (or other sensor) may be fabricated. In another specific example, a temperature sensor 540 (or other sensor) may be attached to a user’s finger (e.g., using adhesives, wraps, clamps, spring loaded clamps, etc.). In this case, the sensor may be wired to another computing device, such as a wrist worn computing device that reads the temperature sensor 540 (or other sensor). In other examples, a wearable device 504 that includes additional sensors and processing functionality may be fabricated.

The housing 505 may include one or more housing components. The housing 505 may include an outer housing 505-b component (e.g., a shell) and an inner housing 505-a component (e.g., a molding). The housing 505 may include additional components (e.g., additional layers) not explicitly illustrated in FIG. 5. For example, in some implementations, the wearable device 504 may include one or more insulating layers that electrically insulate the device electronics and other conductive materials (e.g., electrical traces) from the outer housing 505-b. The housing 505 may provide structural support for the device electronics, battery 511, substrate(s), and other components. For example, the housing 505 may protect the device electronics, battery 511, and substrate(s) from mechanical forces, such as pressure and impacts. The housing 505 may also protect the device electronics, battery 511, and substrate(s) from water and/or other chemicals.

The inner housing 505-a may be configured to interface with the user’s finger. The inner housing 505-a may be formed from a polymer (e.g., a medical grade polymer) or other material. In some implementations, the inner housing 505-a may be transparent. For example, the inner housing 505-a may be transparent to light emitted by the PPG LEDs. In some implementations, the inner housing 505-a component may be molded onto the outer housing 505-b. For example, the inner housing 505-a may include a polymer that is molded (e.g., injection molded) to fit into an outer housing 505-b metallic shell.

The inner housing 505-a and the outer housing 505-b may be fabricated from one or more materials. In some implementations, the inner housing 505-a, the outer housing 505-b, or both, may include a metal, such as titanium, that may provide strength and abrasion resistance at a relatively light weight. Additionally, or alternatively, the inner housing 505-a, and/or the outer housing 505-b may also be fabricated from other materials, such polymers, plastic materials, epoxy materials, ceramic materials, and the like. In some implementations, the outer housing 505-b may be protective as well as decorative.

The wearable device 504 may include one or more substrates (not illustrated). The device electronics and battery 511 may be included on the one or more substrates. For example, the device electronics and battery 511 may be mounted on one or more substrates. Example substrates may include one or more printed circuit boards (PCBs), such as flexible PCB (e.g., polyimide). In some implementations, the electronics/battery 511 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 511 to the device electronics.

The device electronics, battery 511, and substrates may be arranged in the wearable device 504 in a variety of ways. In some implementations, one substrate that includes device electronics may be mounted along the bottom of the wearable device 504 (e.g., the bottom half), such that the sensors (e.g., PPG system 535, temperature sensors 540, motion sensors 545, and other sensors) interface with the underside of the user’s finger. In these implementations, the battery 511 may be included along the top portion of the wearable device 504 (e.g., on another substrate).

The various components/modules of the wearable device 504 represent functionality (e.g., circuits and other components) that may be included in the wearable device 504. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 515 (memory module) of the wearable device 504 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 515 may store any of the data described herein. For example, the memory 515 may be configured to store data (e.g., motion data, temperature data, PPG data) collected by the respective sensors and PPG system 535. Furthermore, memory 515 may include instructions that, when executed by one or more processing circuits, cause the modules to perform various functions attributed to the modules herein. The device electronics of the wearable device 504 described herein are only example device electronics. As such, the types of electronic components used to implement the device electronics may vary based on design considerations.

The functions attributed to the modules of the wearable device 504 (e.g., wearable ring device) described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 530-a of the wearable device 504 may include one or more processors (e.g., processing units), microcontrollers, digital signal processors, systems on a chip (SOCs), and/or other processing devices. The processing module 530-a communicates with the modules included in the wearable device 504. For example, the processing module 530-a may transmit/receive data to/from the modules and other components of the wearable device 504, such as the sensors. As described herein, the modules may be implemented by various circuit components. Accordingly, the modules may also be referred to as circuits (e.g., a communication circuit and power circuit).

The processing module 530-a may communicate with the memory 515. The memory 515 may include computer-readable instructions that, when executed by the processing module 530-a, cause the processing module 530-a to perform the various functions attributed to the processing module 530-a herein. In some implementations, the processing module 530-a (e.g., a microcontroller) may include additional features associated with other modules, such as communication functionality provided by the communication module 520-a (e.g., an integrated Bluetooth Low Energy transceiver) and/or additional onboard memory 515.

The communication module 520-a may include circuits that provide wireless and/or wired communication with the user device 506 (e.g., communication module 520-b of the user device 506). In some implementations, the communication modules 520-a, 520-b may include wireless communication circuits, such as Bluetooth circuits and/or Wi-Fi circuits. In some implementations, the communication modules 520-a, 520-b can include wired communication circuits, such as Universal Serial Bus (USB) communication circuits. Using the communication module 520-a, the wearable device 504 and the user device 506 may be configured to communicate with each other. The processing module 530-a of the ring may be configured to transmit/receive data to/from the user device 506 via the communication module 520-a. Example data may include, but is not limited to, motion data, temperature data, pulse waveforms, heart rate data, HRV data, PPG data, and status updates (e.g., charging status, battery charge level, and/or wearable device 504 configuration settings). The processing module 530-a of the ring may also be configured to receive updates (e.g., software/firmware updates) and data from the user device 506.

The wearable device 504 may include a battery 511 (e.g., a rechargeable battery 511). An example battery 511 may include a Lithium-Ion or Lithium-Polymer type battery 511, although a variety of battery 511 options are possible. The battery 511 may be wirelessly charged. In some implementations, the wearable device 504 may include a power source other than the battery 511, such as a capacitor. The power source (e.g., battery 511 or capacitor) may have a curved geometry that matches the curve of the wearable device 504. In some aspects, a charger or other power source may include additional sensors that may be used to collect data in addition to, or that supplements, data collected by the wearable device 504 itself. Moreover, a charger or other power source for the wearable device 504 may function as a user device 506, in which case the charger or other power source for the wearable device 504 may be configured to receive data from the wearable device 504, store and/or process data received from the wearable device 504, and communicate data between the wearable device 504 and the servers 510.

In some aspects, the wearable device 504 includes a power module 525 that may control charging of the battery 511. For example, the power module 525 may interface with an external wireless charger that charges the battery 511 when interfaced with the wearable device 504. The charger may include a datum structure that mates with a wearable device 504 datum structure to create a specified orientation with the wearable device 504 during charging. The power module 525 may also regulate voltage(s) of the device electronics, regulate power output to the device electronics, and monitor the state of charge of the battery 511. In some implementations, the battery 511 may include a protection circuit module (PCM) that protects the battery 511 from high current discharge, over voltage during charging, and under voltage during discharge. The power module 525 may also include electro-static discharge (ESD) protection.

The one or more temperature sensors 540 may be electrically coupled to the processing module 530-a. The temperature sensor 540 may be configured to generate a temperature signal (e.g., temperature data) that indicates a temperature read or sensed by the temperature sensor 540. The processing module 530-a may determine a temperature of the user in the location of the temperature sensor 540. For example, in the wearable device 504, temperature data generated by the temperature sensor 540 may indicate a temperature of a user at the user’s finger (e.g., skin temperature). In some implementations, the temperature sensor 540 may contact the user’s skin. In other implementations, a portion of the housing 505 (e.g., the inner housing 505-a) may form a barrier (e.g., a thin, thermally conductive barrier) between the temperature sensor 540 and the user’s skin. In some implementations, portions of the wearable device 504 configured to contact the user’s finger may have thermally conductive portions and thermally insulative portions. The thermally conductive portions may conduct heat from the user’s finger to the temperature sensors 540. The thermally insulative portions may insulate portions of the wearable device 504 (e.g., the temperature sensor 540) from ambient temperature.

In some implementations, the temperature sensor 540 may generate a digital signal (e.g., temperature data) that the processing module 530-a may use to determine the temperature. As another example, in cases where the temperature sensor 540 includes a passive sensor, the processing module 530-a (or a temperature sensor 540 module) may measure a current/voltage generated by the temperature sensor 540 and determine the temperature based on the measured current/voltage. Example temperature sensors 540 may include a thermistor, such as a negative temperature coefficient (NTC) thermistor, or other types of sensors including resistors, transistors, diodes, and/or other electrical/electronic components.

The processing module 530-a may sample the user’s temperature over time. For example, the processing module 530-a may sample the user’s temperature according to a sampling rate. An example sampling rate may include one sample per second, although the processing module 530-a may be configured to sample the temperature signal at other sampling rates that are higher or lower than one sample per second. In some implementations, the processing module 530-a may sample the user’s temperature continuously throughout the day and night. Sampling at a sufficient rate (e.g., one sample per second) throughout the day may provide sufficient temperature data for analysis described herein.

The processing module 530-a may store the sampled temperature data in memory 515. In some implementations, the processing module 530-a may process the sampled temperature data. For example, the processing module 530-a may determine average temperature values over a period of time. In one example, the processing module 530-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 515 may store the average temperature values over time. In some implementations, the memory 515 may store average temperatures (e.g., one per minute) instead of sampled temperatures in order to conserve memory 515.

The sampling rate, which may be stored in memory 515, may be configurable. In some implementations, the sampling rate may be the same throughout the day and night. In other implementations, the sampling rate may be changed throughout the day/night. In some implementations, the wearable device 504 may filter/reject temperature readings, such as large spikes in temperature that are not indicative of physiological changes (e.g., a temperature spike from a hot shower). In some implementations, the wearable device 504 may filter/reject temperature readings that may not be reliable due to other factors, such as excessive motion during exercise (e.g., as indicated by a motion sensor 545).

The wearable device 504 (e.g., communication module) may transmit the sampled and/or average temperature data to the user device 506 for storage and/or further processing. The user device 506 may transfer the sampled and/or average temperature data to the server 510 for storage and/or further processing.

Although the wearable device 504 is illustrated as including a single temperature sensor 540, the wearable device 504 may include multiple temperature sensors 540 in one or more locations, such as arranged along the inner housing 505-a near the user’s finger. In some implementations, the temperature sensors 540 may be stand-alone temperature sensors 540. Additionally, or alternatively, one or more temperature sensors 540 may be included with other components (e.g., packaged with other components), such as with the accelerometer and/or processor.

The processing module 530-a may acquire and process data from multiple temperature sensors 540 in a similar manner described with respect to a single temperature sensor 540. For example, the processing module 530 may individually sample, average, and store temperature data from each of the multiple temperature sensors 540. In other examples, the processing module 530-a may sample the sensors at different rates and average/store different values for the different sensors. In some implementations, the processing module 530-a may be configured to determine a single temperature based on the average of two or more temperatures determined by two or more temperature sensors 540 in different locations on the finger.

The temperature sensors 540 on the wearable device 504 (e.g., wearable ring device) may acquire distal temperatures at the user’s finger (e.g., any finger). For example, one or more temperature sensors 540 on the wearable device 504 may acquire a user’s temperature from the underside of a finger or at a different location on the finger. In some implementations, the wearable device 504 may continuously acquire distal temperature (e.g., at a sampling rate). Although distal temperature measured by a wearable device 504 at the finger is described herein, other devices may measure temperature at the same/different locations. In some cases, the distal temperature measured at a user’s finger may differ from the temperature measured at a user’s wrist or other external body location. Additionally, the distal temperature measured at a user’s finger (e.g., a “shell” temperature) may differ from the user’s core temperature. As such, the wearable device 504 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The wearable device 504 may include a PPG system 535. The PPG system 535 may include one or more optical transmitters that transmit light. The PPG system 535 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user’s finger. The PPG signal generated by the PPG system 535 may indicate the perfusion of blood in the illuminated region. For example, the PPG signal may indicate blood volume changes in the illuminated region caused by a user’s pulse pressure. The processing module 530-a may sample the PPG signal and determine a user’s pulse waveform based on the PPG signal. The processing module 530-a may determine a variety of physiological parameters based on the user’s pulse waveform, such as a user’s respiratory rate, heart rate, HRV, oxygen saturation, and other circulatory parameters.

In some implementations, the PPG system 535 may be configured as a reflective PPG system 535 where the optical receiver(s) receive transmitted light that is reflected through the region of the user’s finger. In some implementations, the PPG system 535 may be configured as a transmissive PPG system 535 where the optical transmitter(s) and optical receiver(s) are arranged opposite to one another, such that light is transmitted directly through a portion of the user’s finger to the optical receiver(s).

The number and ratio of transmitters and receivers included in the PPG system 535 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 535.

The PPG system 535 illustrated in FIG. 5 may include a reflective PPG system 535 in some implementations. In these implementations, the PPG system 535 may include a centrally located optical receiver (e.g., at the bottom of the wearable device 504) and two optical transmitters located on each side of the optical receiver. In this implementation, the PPG system 535 (e.g., optical receiver) may generate the PPG signal based on light received from one or both of the optical transmitters. In other implementations, other placements, combinations, and/or configurations of one or more optical transmitters and/or optical receivers are contemplated.

The processing module 530-a may control one or both of the optical transmitters to transmit light while sampling the PPG signal generated by the optical receiver. In some implementations, the processing module 530-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 535 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 530-a may store the pulse waveform in memory 515 in some implementations. The processing module 530-a may process the pulse waveform as it is generated and/or from memory 515 to determine user physiological parameters described herein.

The processing module 530-a may determine the user’s heart rate based on the pulse waveform. For example, the processing module 530-a may determine heart rate (e.g., in beats per minute) based on the time between peaks in the pulse waveform. The time between peaks may be referred to as an interbeat interval (IBI). The processing module 530-a may store the determined heart rate values and IBI values in memory 515.

The processing module 530-a may determine HRV over time. For example, the processing module 530-a may determine HRV based on the variation in the IBIs. The processing module 530-a may store the HRV values over time in the memory 515. Moreover, the processing module 530-a may determine the user’s respiratory rate over time. For example, the processing module 530-a may determine respiratory rate based on frequency modulation, amplitude modulation, or baseline modulation of the user’s IBI values over a period of time. Respiratory rate may be calculated in breaths per minute or as another breathing rate (e.g., breaths per 30 seconds). The processing module 530-a may store user respiratory rate values over time in the memory 515.

The wearable device 504 may include one or more motion sensors 545, such as one or more accelerometers (e.g., 6-D accelerometers) and/or one or more gyroscopes (gyros). The motion sensors 545 may generate motion signals that indicate motion of the sensors. For example, the wearable device 504 may include one or more accelerometers that generate acceleration signals that indicate acceleration of the accelerometers. As another example, the wearable device 504 may include one or more gyro sensors that generate gyro signals that indicate angular motion (e.g., angular velocity) and/or changes in orientation. The motion sensors 545 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 530-a may sample the motion signals at a sampling rate (e.g., 50Hz) and determine the motion of the wearable device 504 based on the sampled motion signals. For example, the processing module 530-a may sample acceleration signals to determine acceleration of the wearable device 504. As another example, the processing module 530-a may sample a gyro signal to determine angular motion. In some implementations, the processing module 530-a may store motion data in memory 515. Motion data may include sampled motion data as well as motion data that is calculated based on the sampled motion signals (e.g., acceleration and angular values).

The wearable device 504 may store a variety of data described herein. For example, the wearable device 504 may store temperature data, such as raw sampled temperature data and calculated temperature data (e.g., average temperatures). As another example, wearable device 504 may store PPG signal data, such as pulse waveforms and data calculated based on the pulse waveforms (e.g., heart rate values, IBI values, HRV values, and respiratory rate values). The wearable device 504 may also store motion data, such as sampled motion data that indicates linear and angular motion.

The wearable device 504, or other computing device, may calculate and store additional values based on the sampled/calculated physiological data. For example, the processing module 530 may calculate and store various metrics, such as sleep metrics (e.g., a Sleep Score), activity metrics, and readiness metrics. In some implementations, additional values/metrics may be referred to as “derived values.” The wearable device 504, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the wearable device 504 is oriented on the user’s finger and if the wearable device 504 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 530-a may compress the data stored in memory 515. For example, the processing module 530-a may delete sampled data after making calculations based on the sampled data. As another example, the processing module 530-a may average data over longer periods of time in order to reduce the number of stored values. In a specific example, if average temperatures for a user over one minute are stored in memory 515, the processing module 530-a may calculate average temperatures over a five minute time period for storage, and then subsequently erase the one minute average temperature data. The processing module 530-a may compress data based on a variety of factors, such as the total amount of used/available memory 515 and/or an elapsed time since the wearable device 504 last transmitted the data to the user device 506.

Although a user’s physiological parameters may be measured by sensors included on a wearable device 504, other devices may measure a user’s physiological parameters. For example, although a user’s temperature may be measured by a temperature sensor 540 included in a wearable device 504, other devices may measure a user’s temperature. In some examples, other wearable devices (e.g., wrist devices) may include sensors that measure user physiological parameters. Additionally, medical devices, such as external medical devices (e.g., wearable medical devices) and/or implantable medical devices, may measure a user’s physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the wearable device 504 can make physiological measurements in a resting/sleep state in order to acquire cleaner physiological signals. In one example, the wearable device 504 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the wearable device 504 may be configured to collect, store, and/or process data, and may transfer any of the data described herein to the user device 506 for storage and/or processing. In some aspects, the user device 506 includes a wearable application 550, an operating system 585 (OS), a web browser application (e.g., web browser 580), one or more additional applications, and a GUI 575. The user device 506 may further include other modules and components, including sensors, audio devices, haptic feedback devices, and the like. The wearable application 550 may include an example of an application (e.g., “app”) that may be installed on the user device 506. The wearable application 550 may be configured to acquire data from the wearable device 504, store the acquired data, and process the acquired data as described herein. For example, the wearable application 550 may include a user interface (UI) module 555, an acquisition module 560, a processing module 530-b, a communication module 520-b, and a storage module (e.g., database 565) configured to store application data.

In some cases, the wearable device 504 and the user device 506 may be included within (or make up) the same device. For example, in some cases, the wearable device 504 may be configured to execute the wearable application 550, and may be configured to display data via the GUI 575.

The various data processing operations described herein may be performed by the wearable device 504, the user device 506, the servers 510, or any combination thereof. For example, in some cases, data collected by the wearable device 504 may be pre-processed and transmitted to the user device 506. In this example, the user device 506 may perform some data processing operations on the received data, may transmit the data to the servers 510 for data processing, or both. For instance, in some cases, the user device 506 may perform processing operations that require relatively low processing power and/or operations that require a relatively low latency, whereas the user device 506 may transmit the data to the servers 510 for processing operations that require relatively high processing power and/or operations that may allow relatively higher latency.

In some aspects, the wearable device 504 (e.g., wearable ring device), user device 506, and server 510 of the system 500 may be configured to evaluate sleep patterns for a user. In particular, the respective components of the system 500 may be used to collect data from a user via the wearable device 504, and generate one or more scores (e.g., Sleep Score, Readiness Score) for the user based on the collected data. For example, as noted previously herein, the wearable device 504 of the system 500 may be worn by a user to collect data from the user, including temperature, heart rate, HRV, and the like. Data collected by the wearable device 504 may be used to determine when the user is asleep in order to evaluate the user’s sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the wearable device 504 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 500 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user’s overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user’s sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user’s overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user’s needs. Typically, adults need 7–9 hours of sleep a night to stay healthy, alert, and to perform at their best both mentally and physically. However, it is normal to have an occasional night of bad sleep, so the sleep balance contributor takes into account long-term sleep patterns to determine whether each user’s sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user’s resting heart rate to stabilize during the night. A sign of a very good recovery is that the user’s resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user’s highest temperature during the nap is at least 0.5°C higher than the highest temperature during the longest period. In some aspects, the ring may measure a user’s body temperature while the user is asleep, and the system 500 may display the user’s average temperature relative to the user’s baseline temperature. If a user’s body temperature is outside of their normal range (e.g., clearly above or below 0.0), the body temperature contributor may be highlighted (e.g., go to a “Pay attention” state) or otherwise generate an alert for the user.

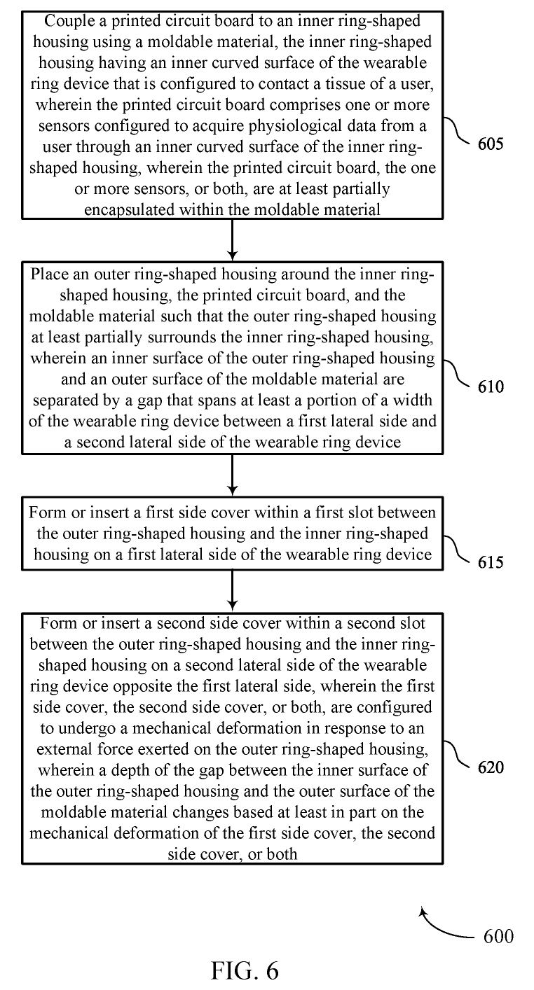

FIG. 6 shows a flowchart illustrating a method 600 that supports shock-absorbing wearable ring structures in accordance with aspects of the present disclosure. For example, the operations of the method 600 may be performed to manufacture a wearable device as described with reference to FIGS. 1–5.

At 605, the method may include coupling a PCB to an inner ring-shaped housing using a moldable material, the inner ring-shaped housing having an inner curved surface of the wearable ring device that is configured to contact a tissue of a user, wherein the PCB comprises one or more sensors configured to acquire physiological data from the user through an inner curved surface of the inner ring-shaped housing, wherein the PCB, the one or more sensors, or both, are at least partially encapsulated within the moldable material. The operations of 605 may be performed in accordance with examples as disclosed herein.

At 610, the method may include placing an outer ring-shaped housing around the inner ring-shaped housing, the PCB, and the moldable material such that the outer ring-shaped housing at least partially surrounds the inner ring-shaped housing, wherein an inner surface of the outer ring-shaped housing and an outer surface of the moldable material are separated by a gap that spans at least a portion of a width of the wearable ring device between a first lateral side and a second lateral side of the wearable ring device. The operations of 610 may be performed in accordance with examples as disclosed herein.

At 615, the method may include forming or inserting a first side cover within a first slot between the outer ring-shaped housing and the inner ring-shaped housing on the first lateral side of the wearable ring device. The operations of 615 may be performed in accordance with examples as disclosed herein.

At 620, the method may include forming or inserting a second side cover within a second slot between the outer ring-shaped housing and the inner ring-shaped housing on the second lateral side of the wearable ring device opposite the first lateral side, wherein the first side cover, the second side cover, or both, are configured to undergo a mechanical deformation in response to an external force exerted on the outer ring-shaped housing, wherein a depth of the gap between the inner surface of the outer ring-shaped housing and the outer surface of the moldable material changes based at least in part on the mechanical deformation of the first side cover, the second side cover, or both. The operations of 620 may be performed in accordance with examples as disclosed herein. The method supports shock-absorbing wearable ring structures which are able to protect the internal components and sensors from damage from external forces.

It should be noted that the methods described above describe possible implementations, and that the operations and the steps may be rearranged or otherwise modified and that other implementations are possible. Furthermore, aspects from two or more of the methods may be combined.

The following provides an overview of aspects of the present disclosure:

Aspect 1: A wearable ring device, comprising: an inner ring-shaped housing having an inner curved surface of the wearable ring device, the inner curved surface configured to at least partially contact a tissue of a user; one or more sensors configured to acquire physiological data from the user through the inner curved surface, wherein the one or more sensors are at least partially encapsulated within a moldable material and coupled with the inner ring-shaped housing via the moldable material; an outer ring-shaped housing having an outer curved surface of the wearable ring device, wherein the outer ring-shaped housing at least partially surrounds the inner ring-shaped housing, wherein an inner surface of the outer ring-shaped housing and an outer surface of the moldable material are separated by a gap that spans at least a portion of a width of the wearable ring device between a first lateral side and a second lateral side of the wearable ring device; and a first side cover and a second side cover at least partially disposed between the outer ring-shaped housing and the inner ring-shaped housing on the first lateral side and the second lateral side of the wearable ring device, respectively, wherein the first side cover, the second side cover, or both, are configured to undergo a mechanical deformation in response to an external force exerted on the outer ring-shaped housing, wherein a depth of the gap between the inner surface of the outer ring-shaped housing and the outer surface of the moldable material changes based at least in part on the mechanical deformation of the first side cover, the second side cover, or both.

Aspect 2: The wearable ring device of aspect 1, wherein the gap spans a full circumference of the wearable ring device between the inner surface of the outer ring-shaped housing and the outer surface of the moldable material.

Aspect 3: The wearable ring device of any of aspects 1 through 2, wherein the first side cover, the second side cover, or both, are configured to undergo the mechanical deformation to dissipate at least a portion of the external force exerted on the outer ring-shaped housing.

Aspect 4: The wearable ring device of any of aspects 1 through 3, wherein the first side cover, the second side cover, or both, are configured to undergo the mechanical deformation to disperse at least a portion of the external force from the outer ring-shaped housing to the inner ring-shaped housing.

Aspect 5: The wearable ring device of any of aspects 1 through 4, wherein the first side cover, the second side cover, or both, comprise ring-shaped fittings that are inserted between the inner ring-shaped housing and the outer ring-shaped housing, an adhesive material that is molded or cured between the inner ring-shaped housing and the outer ring-shaped housing, or both.

Aspect 6: The wearable ring device of any of aspects 1 through 5, wherein the outer ring-shaped housing is coupled to the wearable ring device via a first contact point with the first side cover and a second contact point with the second side cover, the gap spans the portion of the width of the wearable ring device between the first contact point and the second contact point.

Aspect 7: The wearable ring device of aspect 6, wherein the outer ring-shaped housing contacts a first portion of the moldable material proximate to the first lateral side via the first contact point, and a second portion of the moldable material proximate to the second lateral side via the second contact point.

Aspect 8: The wearable ring device of any of aspects 1 through 7, wherein the outer ring-shaped housing comprises a non-conductive material, the wearable ring device further comprising: an antenna configured to transmit and receive wireless communications through the outer ring-shaped housing of the wearable ring device, the antenna at least partially encapsulated within the moldable material, the antenna comprising: an antenna ground plane coupled with the inner ring-shaped housing; and a radiator communicatively coupled with the antenna ground plane, the radiator positioned between the antenna ground plane and the outer surface of the moldable material.

Aspect 9: The wearable ring device of any of aspects 1 through 8, further comprising: a curved battery that is at least partially encapsulated within the moldable material such that the gap separates an outer surface of the curved battery and the outer surface of the moldable material from the inner surface of the outer ring-shaped housing.

Aspect 10: The wearable ring device of any of aspects 1 through 9, wherein the outer ring-shaped housing comprises a non-deformable material.

Aspect 11: The wearable ring device of aspect 10, wherein the non-deformable material comprises a ceramic material.

Aspect 12: The wearable ring device of any of aspects 1 through 11, further comprising: a compressible material, an insulating material, or both, that fills at least a portion of the gap.

Aspect 13: A method for manufacturing a wearable ring device, comprising: coupling a PCB to an inner ring-shaped housing using a moldable material, the inner ring-shaped housing having an inner curved surface of the wearable ring device that is configured to contact a tissue of a user, wherein the PCB comprises one or more sensors configured to acquire physiological data from the user through an inner curved surface of the inner ring-shaped housing, wherein the PCB, the one or more sensors, or both, are at least partially encapsulated within the moldable material; placing an outer ring-shaped housing around the inner ring-shaped housing, the PCB, and the moldable material such that the outer ring-shaped housing at least partially surrounds the inner ring-shaped housing, wherein an inner surface of the outer ring-shaped housing and an outer surface of the moldable material are separated by a gap that spans at least a portion of a width of the wearable ring device between a first lateral side and a second lateral side of the wearable ring device; forming or inserting a first side cover within a first slot between the outer ring-shaped housing and the inner ring-shaped housing on the first lateral side of the wearable ring device; and forming or inserting a second side cover within a second slot between the outer ring-shaped housing and the inner ring-shaped housing on the second lateral side of the wearable ring device opposite the first lateral side, wherein the first side cover, the second side cover, or both, are configured to undergo a mechanical deformation in response to an external force exerted on the outer ring-shaped housing, wherein a depth of the gap between the inner surface of the outer ring-shaped housing and the outer surface of the moldable material changes based at least in part on the mechanical deformation of the first side cover, the second side cover, or both.

Aspect 14: The method of aspect 13, wherein the gap spans a full circumference of the wearable ring device between the inner surface of the outer ring-shaped housing and the outer surface of the moldable material.

Aspect 15: The method of any of aspects 13 through 14, further comprising: filling at least a portion of the gap with a compressible material, an insulating material, or both.

Aspect 16: The method of any of aspects 13 through 15, further comprising: coupling a curved battery with the PCB, wherein the PCB and the curved battery are coupled with the inner ring-shaped housing by at least partially encapsulating the PCB and the curved battery within the moldable material.

Aspect 17: The method of any of aspects 13 through 16, wherein forming or inserting the first side cover and the second side cover comprises: inserting a first ring-shaped fitting within the first slot between the inner ring-shaped housing and the outer ring-shaped housing, wherein the first side cover comprises the first ring-shaped fitting; and inserting a second ring-shaped fitting within the second slot between the inner ring-shaped housing and the outer ring-shaped housing, wherein the second side cover comprises the second ring-shaped fitting, wherein the first ring-shaped fitting and the second ring-shaped fitting extend around a full circumference of the wearable ring device on the first lateral side and the second lateral side of the wearable ring device, respectively.

Aspect 18: The method of any of aspects 13 through 17, wherein forming or inserting the first side cover and the second side cover comprises: curing an adhesive material within the first slot and the second slot, wherein the first side cover and the second side cover comprise the adhesive material.

Aspect 19: A wearable ring device manufactured by the method of any of aspects 13 through 18.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
an inner ring-shaped housing having an inner curved surface of the wearable ring device, the inner curved surface configured to at least partially contact a tissue of a user;
one or more sensors configured to acquire physiological data from the user through the inner curved surface, wherein the one or more sensors are at least partially encapsulated within a moldable material and coupled with the inner ring-shaped housing via the moldable material;
an outer ring-shaped housing having an outer curved surface of the wearable ring device, wherein the outer ring-shaped housing at least partially surrounds the inner ring-shaped housing, wherein an inner surface of the outer ring-shaped housing and an outer surface of the moldable material are separated by a gap that spans at least a portion of a width of the wearable ring device between a first lateral side and a second lateral side of the wearable ring device; and
a first side cover and a second side cover at least partially disposed between the outer ring-shaped housing and the inner ring-shaped housing on the first lateral side and the second lateral side of the wearable ring device, respectively, wherein the first side cover, the second side cover, or both, are configured to undergo a mechanical deformation in response to an external force exerted on the outer ring-shaped housing, wherein a depth of the gap between the inner surface of the outer ring-shaped housing and the outer surface of the moldable material changes based at least in part on the mechanical deformation of the first side cover, the second side cover, or both.

2. The wearable ring device of claim 1, wherein the gap spans a full circumference of the wearable ring device between the inner surface of the outer ring-shaped housing and the outer surface of the moldable material.

3. The wearable ring device of claim 1, wherein the first side cover, the second side cover, or both, are configured to undergo the mechanical deformation to dissipate at least a portion of the external force exerted on the outer ring-shaped housing.

4. The wearable ring device of claim 1, wherein the first side cover, the second side cover, or both, are configured to undergo the mechanical deformation to disperse at least a portion of the external force from the outer ring-shaped housing to the inner ring-shaped housing.

5. The wearable ring device of claim 1, wherein the first side cover, the second side cover, or both, comprise ring-shaped fittings that are inserted between the inner ring-shaped housing and the outer ring-shaped housing, an adhesive material that is molded or cured between the inner ring-shaped housing and the outer ring-shaped housing, or both.

6. The wearable ring device of claim 1, wherein the outer ring-shaped housing is coupled to the wearable ring device via a first contact point with the first side cover and a second contact point with the second side cover, wherein the gap spans the portion of the width of the wearable ring device between the first contact point and the second contact point.

7. The wearable ring device of claim 6, wherein the outer ring-shaped housing contacts a first portion of the moldable material proximate to the first lateral side via the first contact point, and a second portion of the moldable material proximate to the second lateral side via the second contact point.

8. The wearable ring device of claim 1, wherein the outer ring-shaped housing comprises a non-conductive material, the wearable ring device further comprising:
an antenna configured to transmit and receive wireless communications through the outer ring-shaped housing of the wearable ring device, the antenna at least partially encapsulated within the moldable material, the antenna comprising:
an antenna ground plane coupled with the inner ring-shaped housing; and
a radiator communicatively coupled with the antenna ground plane, the radiator positioned between the antenna ground plane and the outer surface of the moldable material.

9. The wearable ring device of claim 1, further comprising:
a curved battery that is at least partially encapsulated within the moldable material such that the gap separates an outer surface of the curved battery and the outer surface of the moldable material from the inner surface of the outer ring-shaped housing.

10. The wearable ring device of claim 1, wherein the outer ring-shaped housing comprises a non-deformable material.

11. The wearable ring device of claim 10, wherein the non-deformable material comprises a ceramic material.

12. The wearable ring device of claim 1, further comprising:
a compressible material, an insulating material, or both, that fills at least a portion of the gap.

13. A method for manufacturing a wearable ring device, comprising:
coupling a printed circuit board to an inner ring-shaped housing using a moldable material, the inner ring-shaped housing having an inner curved surface of the wearable ring device that is configured to contact a tissue of a user, wherein the printed circuit board comprises one or more sensors configured to acquire physiological data from the user through an inner curved surface of the inner ring-shaped housing, wherein the printed circuit board, the one or more sensors, or both, are at least partially encapsulated within the moldable material;
placing an outer ring-shaped housing around the inner ring-shaped housing, the printed circuit board, and the moldable material such that the outer ring-shaped housing at least partially surrounds the inner ring-shaped housing, wherein an inner surface of the outer ring-shaped housing and an outer surface of the moldable material are separated by a gap that spans at least a portion of a width of the wearable ring device between a first lateral side and a second lateral side of the wearable ring device;
forming or inserting a first side cover within a first slot between the outer ring-shaped housing and the inner ring-shaped housing on the first lateral side of the wearable ring device; and
forming or inserting a second side cover within a second slot between the outer ring-shaped housing and the inner ring-shaped housing on the second lateral side of the wearable ring device opposite the first lateral side, wherein the first side cover, the second side cover, or both, are configured to undergo a mechanical deformation in response to an external force exerted on the outer ring-shaped housing, wherein a depth of the gap between the inner surface of the outer ring-shaped housing and the outer surface of the moldable material changes based at least in part on the mechanical deformation of the first side cover, the second side cover, or both.

14. The method of claim 13, wherein the gap spans a full circumference of the wearable ring device between the inner surface of the outer ring-shaped housing and the outer surface of the moldable material.

15. The method of claim 13, further comprising:
filling at least a portion of the gap with a compressible material, an insulating material, or both.

16. The method of claim 13, further comprising:
coupling a curved battery with the printed circuit board, wherein the printed circuit board and the curved battery are coupled with the inner ring-shaped housing by at least partially encapsulating the printed circuit board and the curved battery within the moldable material.

17. The method of claim 13, wherein forming or inserting the first side cover and the second side cover comprises:
inserting a first ring-shaped fitting within the first slot between the inner ring-shaped housing and the outer ring-shaped housing, wherein the first side cover comprises the first ring-shaped fitting; and
inserting a second ring-shaped fitting within the second slot between the inner ring-shaped housing and the outer ring-shaped housing, wherein the second side cover comprises the second ring-shaped fitting, wherein the first ring-shaped fitting and the second ring-shaped fitting extend around a full circumference of the wearable ring device on the first lateral side and the second lateral side of the wearable ring device, respectively.

18. The method of claim 13, wherein forming or inserting the first side cover and the second side cover comprises:
curing an adhesive material within the first slot and the second slot, wherein the first side cover and the second side cover comprise the adhesive material.
